(19) United States
(12) Patent Application Publication
(10) Pub. No.: US 2012/0022017 A1
(43) Pub. Date: Jan. 26, 2012

(54) COMPOSITION AND A METHOD THEREOF

(75) Inventors: Villoo Morawala Patell, Karnataka (IN); Henjarappa Jagadeesh Badamaranahalli, Karnataka (IN); Dhruvdev Vyas, Karnataka (IN); Rajesh Ullanat, Karnataka (IN); Parag Shah, Karnataka (IN); Renuka Jain, Karnataka (IN)

(73) Assignee: AVESTHAGEN LIMITED, Bangalore, Karnataka (IN)

(21) Appl. No.: 13/126,652

(22) PCT Filed: Oct. 28, 2009
(86) PCT No.: PCT/IB2009/007249

§ 371 Date: Aug. 3, 2011

(30) Foreign Application Priority Data
Oct. 28, 2008 (IN) 2617/CHE/2008

(51) Int. Cl. A61K 31/736; A61P 3/10

(52) U.S. Cl. 514/54

(57) ABSTRACT

The present invention describes a palatable and orally administrable form of composition comprising proteins, galactomannans and base matrix optionally along with acceptable additives.

### FIELD OF INVENTION

The present invention describes a palatable and orally administrable form of composition comprising proteins, galactomannans and base matrix optionally along with acceptable additives.

### BACKGROUND AND PRIOR ART OF THE INVENTION

Diabetes mellitus is one of the most prevalent degenerative diseases, which is generally characterized by the abnormalities in the body's ability to use sugar. Mainly three forms namely Type 1 diabetes, Type 2 diabetes and Gestational diabetes are known to occur. However, the diabetes epidemic relates particularly to type 2 diabetes and is largely discussed here in the context of trigonella.

Diabetes type 1 is an autoimmune disease that occurs when the body's immune system turns against a part of the body. In diabetes, the immune system attacks the insulin-producing beta cells in the pancreas and destroys them resulting in production of very little or no insulin. Therefore, a person afflicted by type 1 diabetes is dependent on daily dose of insulin. At present, scientists however have not been able to underpin the exact causes for the body's immune system to attack the beta cells, but they believe that autoimmune genetic, and environmental factors are involved. It develops most often in children and young adults, but can appear at any age. The gestational diabetes is however largely observed in pregnant women and is mainly characterized by temporary imbalance of the blood sugar level.

Type 2 diabetes is the most common form of diabetes afflicting approximately 90-95% of the people. This form of diabetes is generally associated with older age, obesity, family history, previous history of gestational diabetes, physical inactivity and ethnicity. About 80% of people with type 2 diabetes are overweight. It is increasingly being diagnosed in children and adolescents. In type 2 diabetes, the pancreases usually produce enough insulin, but the body is unable to use the insulin effectively, resulting in a condition called insulin resistance. After several years, insulin production decreases and the result is the same as for type 1 diabetes i.e., glucose builds up in the blood and the body cannot make efficient use of its main source of fuel. In the long term, diabetes also leads to other related problems like atherosclerosis, hyperlipidemia, retinal damage, neurological damage, and blindness due to spikes in blood sugar in patients during the day.

Currently, there is no cure for diabetes. The use of prescription drugs can help control diabetes symptoms and keep the disease from progressing; however, diabetes patients can also adopt various lifestyle changes to help control the disease.

One of the first lifestyle changes recommended to diabetics by health care practitioners is to follow guidelines for good general health, which includes indulging in regular physical activity, avoiding smoking, maintaining a healthy body weight, consuming a diet rich in whole grains, fruits, vegetables, multivitamin/mineral dietary supplement and omega-3 long-chain polyunsaturated fatty acids (LC-PUFAs), and avoiding saturated fats.

Amongst many approaches gaining popularity are adopting healthy glycemic practices. A lifestyle change, diabetics can pursue is to control the disease by managing blood glucose levels through diet. This is being done by developing and introducing ingredients designed to modulate blood sugar. An important approach towards achieving this is the intake of diet comprising proteins, galactomannans and base matrix along with acceptable additives as a composite extract obtained from the herb Trigonella foenum-graecum, popularly known as fenugreek. The extract is compositely known as TEESTAR™. The method of extraction of active ingredient of the Teestar™ has been explained in detail under patent application No. PCT/IN2007/000580.

The present invention deals with incorporating the extract, Teestar™ in a clinically validated comestible form. This approach can particularly prove beneficial, even for otherwise healthy individuals who are at increased risk of developing diabetes or pre-diabetic patients. Generally, among diabetics, each 1-percentage-point reduction in blood sugar causes a 40-percent reduction in the risk of microvascular complications including eye, kidney and nerve diseases. In general, diets lacking adequate dietary fiber may put individuals at risk of developing Type II diabetes.

The intake of food affects the body's need for insulin and its ability to lower blood sugar. Diet is therefore found to be the cornerstone in diabetes treatment. Research of Paul F. Jacques in American Journal of Clinical Nutrition suggests, whole grains appear to be beneficial with respect to insulin levels and potentially with respect to diabetes risk. Whole-wheat products like crackers have been recommended as part of healthy snacks for diabetic.

### OBJECTIVE OF THE PRESENT INVENTION

The main objective of the present invention is to obtain a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives.

Another main objective of the present invention is to obtain a method of obtaining the said composition.

Yet another main objective of the present invention is to obtain a method of consumption of the said composition.

### STATEMENT OF THE PRESENT INVENTION

Accordingly, the present invention relates to a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives; a method of obtaining a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprising step of mixing of the proteins, the galactomannans, and the ingredients of base matrix followed by sheeting lamination, final gauging, cutting and baking to obtain the composition; and a method of consumption of a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprises consumption of said composition by a user.

### DETAILED DESCRIPTION

The present invention relates to composition comprising of but not limiting to proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives.

In another embodiment of the present invention the composition is a composite extract.

In another embodiment of the present invention the whole wheat flour ranges between 22% and 34%, refined flour ranges between 8% and 12%, wheat bran flour ranges between 8% and 12%, edible vegetable oils ranges between 1% and 4%, fructose ranges between 2% and 7%, salt ranges between 0.5% and 3%, Fibre(Teestar) between 0.5% and 6%, Palmolein between 1% and 4%, Seasoning between 2% and 7%, Rosemary between 0.01% and 2%, Water between 15% and 45% and leavening agents range between 0.1% and 2%.

In yet another embodiment of the present invention the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In still another embodiment of the present invention the leavening agent is yeast, preferably dried baker's yeast.

In still another embodiment of the present invention the composition is thermally stable.

In still another embodiment of the present invention the composition is a comestible.

In still another embodiment of the present invention the comestible is a cracker.

In still another embodiment of the present invention the cracker possesses anti-diabetic property.

In still another embodiment of the present invention the anti-diabetic property is due to blood sugar modulation achieved by lowering of Glycemic Index.

The present invention relates to a method of obtaining a composition comprising of but not limiting to proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprising step of mixing of the proteins, the galactomannans, and the ingredients of base matrix followed by sheeting lamination, final gauging, cutting and baking to obtain the composition.

The present invention relates to a method of consumption of a composition comprising of but not limiting to proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprises consumption of said composition by a user.

In another embodiment of the present invention the whole wheat flour ranges between 22% and 34%, refined flour ranges between 8% and 12%, wheat bran flour ranges between 8% and 12%, edible vegetable oils ranges between 1% and 4%, fructose ranges between 2% and 7%, salt ranges between 0.5% and 3%, Fibre(Teestar) between 0.5% and 6%, Palmolein between 1% and 4%, Seasoning between 2% and 7%, Rosemary between 0.01% and 2%, Water between 15% and 45% and leavening agents range between 0.1% and 2%.

In yet another embodiment of the present invention additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In still another embodiment of the present invention the leavening agent is yeast, preferably dried baker's yeast.

Another embodiment of the present invention relates to developing a base matrix of whole-wheat crackers, which would qualify for its use as vehicle for delivering Teestar™ for blood sugar modulation by lowering Glycemic Index.

Yet another embodiment of the present invention relates to testing thermal stability of the activity of Teestar™ and detailed nutritional evaluation of the base matrix

Still another embodiment of the present invention relates to the product formulation and pilot scale clinical trial of whole-wheat crackers with incorporation of the bioNutritional lead, Teestar™.

The base formulation of Whole-wheat crackers was developed to be used as matrix for incorporation of Teestar™ for blood sugar management. The ingredients used for developing base matrix were, whole-wheat flour, refined flour, wheat bran flakes, edible vegetables oils, fructose, spices and condiments, salt and leavening agent(s). The base product has the uniqueness of having natural leavening agent, no trans fat and high in fibre content.

### Example 1

The base matrix developed was taken forward for detailed nutritional analysis to determine the carbohydrates, protein, total fat, fatty acid profiles, dietary fiber content with quantitative estimations of soluble and insoluble fraction, sodium and cholesterol content.

Table 1 shows detailed nutritional profile of the base product conferring along the base USP of the product.

TABLE 1

| | | Observed |
|---|---|---|
| Sl. No. | Test | values |
| 1 | Protein, % by mass | 11.7 |
| 2 | Carbohydrates (as dextrose), % by mass | 64.5 |
| 3 | Total fat, % by mass | 9 |
| 4 | Saturated fatty acids, % of extracted fat | 17.9 |
| 5 | Mono-unsaturated fatty acids, % of extracted fat | 24.6 |
| 6 | Poly-unsaturated fatty acid, % of extracted fat | 55 |
| 7 | Total dietary fiber, % by mass | 8 |
| 8 | Soluble fiber, % by mass | 0.2 |
| 9 | Insoluble fiber, % by mass | 7.8 |
| 10 | Calorific value, Kcal/100 gm | 385.8 |

The above formulation was used as base matrix for incorporation of the Teestar™ to develop whole-wheat crackers with low Glycemic Index proposition.

### Example 2

The product formulation was initiated by incorporation of bioNutritional lead, Teestar™, at four concentrations 2%, 4%, 6% and 8%. The samples were evaluated for the soluble fiber content and carbohydrates as dextrose equivalent. The control samples without the bioNutritional lead were used as reference.

The formulation with 4% Teestar™ incorporation was checked for its nutritional profile as well as for the concentration of Teestar™ incorporated.

### Example 3

### Thermal Stability of Teestar™

Teestar™ was tested for thermal stability at varying temperature and time. At the end of the treatment, average value of galactomannan content of Teestar™ ingredient was calculated. The results were noted as reflected in Table 2.

TABLE 2

| Particulars | Average Galactomannan % |
|---|---|
| 100 degree C. for 5 min | 60.32 +/− 0.61 |
| 100 degree C. for 10 min | 61.09 +/− 2.0 |
| 100 degree C. for 15 min | 66.05 +/− 2.0 |
| 150 degree C. for 5 min | 57.70 +/− 1.4 |
| 150 degree C. for 10 min | 57.74 +/− 1.5 |
| 150 degree C. for 15 min | 58.40 +/− 1.6 |
| 200 degree C. for 5 min | 60.18 +/− 0.3 |
| 200 degree C. for 10 min | 59.39 +/− 0.8 |
| 200 degree C. for 15 min | 58.53 +/− 0.3 |
| 250 degree C. for 5 min | 63.26 +/− 0.1 |
| 250 degree C. for 10 min | 63.99 +/− 1.8 |
| 250 degree C. for 15 min | 46.76 +/− 1.3 |
| Control | 56.48 +/− 1.5 |

Teestar™ ingredient was found to be stable at temperatures ranging to 250 degree C. for 10 mins under standard testing conditions. Given the thermal stability of Teestar™ ingredient, it was having the property of baking compatibility making it suitable for bakery products.

### Example 4

### Manufacturing of Whole Wheat Crackers with Teestar™

The following steps were involved in manufacturing of the Whole Wheat Cracker incorporated with the composite extract Teestar™:
1. Weigh formulation water. Ensure temperature of water at around 30° C.
2. Weigh fructose, add to water and make a solution.
3. Weigh required quantity of fructose solution and yeast. Add the yeast to the fructose solution and make yeast slurry. Allow the yeast to be activated for at least 15 minutes. The temperature of the solution at this stage would be ambient.
4. Weigh required quantity of Whole Wheat Flour (Atta), Wheat Flour (Maida), Wheat Bran, Fructose, spice mix, Salt and Teestar™. Ensure that the Teestar™ is thoroughly mixed with 10 kg of flour. Charge all the ingredients in the sigma mixer and dry run the mixer.
5. Add Yeast Slurry and required quantity of water to the mixer, and mix.
6. Weigh seasoning and finally add to the dough, mix for 5 minutes.
7. Check the development of the dough ensuring optimum gluten development.
8. Weigh required quantity of Oil and Antioxidants. Mix them thoroughly together.
9. Charge this Oil-Antioxidant solution to the mixer, mix it for 5 minutes and ensure uniform oil dispersion.
10. Sheet the dough as per desired thickness.
11. Cut the sheeted dough as per standard size and shape.
12. Bake the wet crackers in a tunnel oven.
13. Cool the crackers by passing through cooling conveyor.
14. Pack the finished product.

### Example 5

### Taste Enhancement and Nutritional Profile

The process development was carried out with respect to addition of seasoning to incorporate organoleptic acceptance of the product. The seasoning comprises of, but not limiting to, kasuri methi based additives which, in addition to masking bitterness and enhances the taste, making it organoleptically acceptable.

The nutritional profile of the Teestar™ whole-wheat crackers is shown in table 3.

TABLE 3

| Tests | 100 g | 25 g | Test method |
|---|---|---|---|
| Protein (N × 6.25) (g) | 11.94 | 2.985 | IS: 4706 (Part II) 1978 |
| Carbhohydrates (g) | 53.21 | 13.3 | By calculations |
| Fat (Soxhlet) (g) | 9.2 | 2.3 | AOAC 2003.05 |
| Total Dietary Fibre (g) | 17.98 | 4.495 | AOAC 991.43 |
| Soluble dietary fibre (g) | 3.93 | 0.9825 | AOAC 991.43 |
| In-soluble dietary fibre (g) | 14.05 | 3.5125 | AOAC 991.43 |
| Energy (Kcal) | 379.2 | 94.8 | By Calculations |
| Energy From Fat (Kcal) | 82.8 | 20.7 | By calculations |

### Example 6

### Safety and Efficacy Studies

The whole-wheat crackers with Teestar™ were taken for clinical trial with recommendation of 5 crackers per serving which will deliver 1 gm of Teestar™.

The invention is further supported by real-time bioactivity efficacy studies through a systematically conducted pilot scale clinical trial of baked whole-wheat crackers with incorporation of the bioNutritional lead, Teestar™, comparing the effect with Teestar™ capsules as well as Placebo capsules.

An open label, randomized, placebo-controlled, parallel-group, three arm (test 1, test 2 and placebo), multiple dose efficacy study in healthy human subjects. Subjects received the IP twice daily for 7 consecutive days, 20 minutes prior to a standardized meal.

The main selection criteria of subjects understudy were healthy adult males aged between 18 to 45 years (both ages inclusive), who gave informed written consent; subjects weighing as per the standard height and weight; subject whose pre-study screening laboratory tests, X-ray and 12 lead ECG are either normal or within acceptable limits; subject with negative test for drugs of abuse, RPR, Hepatitis B, C, HIV 1 and 2; subject available for the entire study period and capable of understanding and communicating with the investigators and clinical study facility staff.

All subjects enrolled received the investigational products, as per their treatment assignment either to the test arm 1 or test arm 2 or placebo arm, for a period of 7 days. Subjects received either Teestar™ 2×500 mg capsules, Teestar™ 5 crackers or Placebo 2×500 mg capsules twice daily for 7 consecutive days. The sequence of assigning the treatment was determined by a randomization scheme generated using SAS®.

The blood glucose concentrations were evaluated for the test groups in comparison to placebo at all time points during the course of study period. Serum insulin levels were analyzed for 9 randomly selected subjects (3 from each arm) at all time points on day 2 and day 7.

Descriptive statistical analysis and 2-sided t-test were used, as planned in the protocol. Summary statistics of individual and mean blood glucose concentrations in all three treatment arms over all the time points after each of the two dosings in a day, on all 7 days were provided.

The study demonstrates an effective and statistically significant reduction of mean blood glucose levels in Teestar™ fed groups, both in Capsule and Cracker form, in comparison to placebo control at 60 minutes after the morning dose when tested at a 10% level of significance. However the effects shown by the cracker formulation was greater than those achieved by the capsule form as reflected in the data above.

### Results

There were no test product related adverse event(s) reported during the course of the study. Table 4 shows comparative table for the Mean Blood Glucose levels at time points at 60 minutes for Teestar™ cracker Vs Placebo (Morning). Table 5 shows comparative table for the Mean Blood Glucose levels at time points at 60 minutes for Teestar™ capsule Vs Placebo (Morning).

TABLE 4

| Time | | | | | Standard | Mean | | |
|---|---|---|---|---|---|---|---|---|
| Points | Treatment | Session | N | Mean | Error | Difference | 90% Cl | P Value |
| 60 | Teestar ™ | M | 63 | 125.21 | 2.3567 | −9.77 | (−17.23,-2.32) | 0.0121 |
| | Cracker | | | | | | | |
| 60 | Without | M | 56 | 134.98 | 3.1312 | | | |
| | Teestar ™ | | | | | | | |

TABLE 5

| Time | | | | | Standard | Mean | | |
|---|---|---|---|---|---|---|---|---|
| Points | Treatment | Session | N | Mean | Error | Difference | 90% Cl | P Value |
| 60 | Teestar ™ | M | 56 | 127.23 | 2.5608 | −7.75 | (−14.46,-1.04) | 0.0580 |
| | Capsule | | | | | | | |
| 60 | Without | M | 56 | 134.98 | 3.1312 | | | |
| | Teestar ™ | | | | | | | |

The mean blood glucose levels at 60 minutes post morning dose for Teestar™ Capsule [(127.23 mg/dl)] and Teestar™ Cracker [(125.21 mg/dl)] was lower when compared to Placebo [(134.98 mg/dl)]. The mean difference at 60 minutes was −7.75 mg/dl for Teestar™ Capsule and −9.77 mg/dl for Teestar™ Cracker compared to placebo.

Statistical significance was achieved at the 0.1 level of significance with p-values of 0.0580 for Teestar™ Capsule and 0.0121 for Teestar™ Cracker, at 60 minutes after the morning dose respectively.

The study demonstrates an effective and statistically significant reduction of mean blood glucose levels in Teestar™ fed groups, both in Capsule and Cracker form, in comparison to placebo control at 60 minutes after the morning meal. However the effects shown by the cracker formulation was greater than those achieved by the capsule form as reflected in the data above.

Based on the results obtained in this study it can be concluded that the products Teestar™ Crackers manufactured by Avesthagen Limited, India, administered twice daily, 20 minutes prior to consumption of meals, may retard absorption of the carbohydrates from the gut in humans and help in reducing post prandial blood glucose concentrations at 60 minutes after the morning meal. Based on the observations and evaluation of adverse events, clinical laboratory evaluation and vital signs, it can be concluded that Teestar™ incorporated cracker formulation was well tolerated and was found to be safe for human use, in the doses evaluated in this study.

The mechanism of action of Teestar™ crackers in comparison to Teestar™ capsules is different in respect to time points. The data presented in the table above clearly suggests that Cracker with Teestar™ activity is better when compared with Teestar™ in the capsule form.

The clinical study regarding Teestar™ in the cracker form (Galactomannans) showed a significant decrease in glucose absorption in normal adults. With reference to a historical study that was carried out a simultaneous administration of Galactomannans and Psyllium husk (85%) or Oat Bran concentrate (15%), showed a lowering of absorption of approximately 5 mg/dl of glucose (actual Galactomannan concentration 1 gm). Psyllium husk or Oat bran are also known for their inhibition of glucose absorption in the gut.

In this study, the groups of volunteers were administered crackers containing 1 gm of Teestar™ alone and it showed an inhibitory potential of more than 50% in comparison to the earlier mentioned study. From this data it can be concluded that the cracker containing Teestar™ is more potent than other similar formulations available in the market.

Although, the invention is described in detail with reference to specific embodiments, it will be understood that, the variations, which are functionally equivalent, would fall within the scope of this invention. Various modifications of the invention, in addition to those shown and described herein, will become apparent to those skilled in the art from the mentioned description. Such modifications also are intended to fall within the scope of the invention and appended claims.

We claim:

1) A composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives.

2) The composition as claimed in claim 1, wherein the composition is a composite extract.

3) The composition as claimed in claim 1, wherein the whole wheat flour ranges between 22% and 34%, refined flour ranges between 8% and 12%, wheat bran flour ranges between 8% and 12%, edible vegetable oils ranges between 1% and 4%, fructose ranges between 2% and 7%, salt ranges between 0.5% and 3%, Fibre(Teestar) between 0. 5% and 6%, Palmolein between 1% and 4%, Seasoning between 2% and 7%, Rosemary between 0.01% and 2%, Water between 15% and 45% and leavening agents range between 0.1% and 2%.

4) The composition as claimed in claim 1, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

5) The composition as claimed in claim 1, where in the leavening agent is yeast, preferably dried baker's yeast.

6) The composition as claimed in claim 1, wherein the composition is thermally stable.

7) The composition as claimed in claim 1, wherein the composition is a comestible.

8) The composition as claimed in claim 5, wherein the comestible is a cracker.

9) The composition as claimed in claim 6, wherein the cracker possesses anti-diabetic property.

10) The composition as claimed in claim 7, wherein the anti-diabetic property is due to blood sugar modulation achieved by lowering of Glycemic Index.

11) A method of obtaining a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprising step of mixing of the proteins, the galactomannans, and the ingredients of base matrix followed by sheeting lamination, final gauging, cutting and baking to obtain the composition.

12) The method as claimed in claim 10, wherein the whole wheat flour ranges between 22% and 34%, refined flour ranges between 8% and 12%, wheat bran flour ranges between 8% and 12%, edible vegetable oils ranges between 1% and 4%, fructose ranges between 2% and 7%, salt ranges between 0.5% and 3%, Fibre(Teestar) between 0. 5% and 6%, Palmolein between 1% and 4%, Seasoning between 2% and 7%, Rosemary between 0.01% and 2%, Water between 15% and 45% and leavening agents range between 0.1% and 2%.

13) The method as claimed in claim 10, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

14) The method as claimed in claim 10, wherein the leavening agent is yeast, preferably dried baker's yeast.

15) A method of consumption of a composition comprising proteins ranging between 20% w/w to 30% w/w, galactomannans ranging between 32% w/w to 80% w/w and base matrix comprising of whole-wheat flour, refined flour, wheat bran flakes, edible vegetable oils, fructose, spices, condiments, salt and leavening agent(s) optionally along with acceptable additives, said method comprises consumption of said composition by a user.

16) The method as claimed in claim 14, wherein the whole wheat flour ranges between 22% and 34%, refined flour ranges between 8% and 12%, wheat bran flour ranges between 8% and 12%, edible vegetable oils ranges between 1% and 4%, fructose ranges between 2% and 7%, salt ranges between 0.5% and 3%, Fibre(Teestar) between 0. 5% and 6%, Palmolein between 1% and 4%, Seasoning between 2% and 7%, Rosemary between 0.01% and 2%, Water between 15% and 45% and leavening agents range between 0.1% and 2%.

17) The method as claimed in claim 14, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

18) The method as claimed in claim 14, wherein the method of consumption is oral.

19) A composition, a method of obtaining a composition and a method of consumption of a composition as substantially described herein with reference to tables.
